(19) United States
(12) Patent Application Publication
(10) Pub. No.: US 2022/0023575 A1
(43) Pub. Date: Jan. 27, 2022

(54) BREATHING ASSIST DEVICE FOR USE IN MASK

(71) Applicant: OCEAN STAR PROMOTIONS INC., Warwick, RI (US)

(72) Inventor: Kejian CHEN, Yiwu (CN)

(73) Assignee: OCEAN STAR PROMOTIONS INC., Warwick, RI (US)

(21) Appl. No.: 17/037,580

(22) Filed: Sep. 29, 2020

(51) Int. Cl. A61M 16/06; A61M 16/00; A61M 16/10

(52) U.S. Cl. CPC A61M 16/0688; A61M 16/0057; A61M 2205/07; A61M 2205/587; A61M 2205/8206; A61M 16/1055

(57) ABSTRACT

The present disclosure provides a breathing assist device for use in a mask. The breathing assist device may include an air pump, a connecting tube, and a fixing member. A first end of the connecting tube may be disposed on an inner surface of the mask by the fixing member, and a second end of the connecting tube may be connected to the air pump.

### CROSS-REFERENCE TO RELATED APPLICATION

This application claims priority of Chinese Patent Application No. 202021506139.0 filed on Jul. 27, 2020, the entire contents of which are hereby incorporated by reference.

### TECHNICAL FIELD

The present disclosure relates to the technical field of respiratory protection, and in particular, to a breathing assist device for use in a mask.

### BACKGROUND

In daily life, air-filtering masks are widely-used respiratory protection products. People only need to wear the air-filtering masks directly on the mouth and nose to filter the air entering the mouth and nose to block harmful gases, odor, droplets, viruses, and other substances. But for some users, in particular the elderly or people with cardiopulmonary dysfunction, wearing a mask may reduce the amount of air entering the mouth and nose, resulting in breathing difficulties. Therefore, the present disclosure provides breathing assist devices for use in masks to solve the problem of breathing difficulties of users and convenient to carry.

### SUMMARY

In one aspect of the present disclosure, a breathing assist device for use in a mask is provided. The breathing assist device may include an air pump, a connecting tube, and a fixing member. A first end of the connecting tube may be disposed on an inner surface of the mask by the fixing member, and a second end of the connecting tube may be connected to the air pump.

In some embodiments, a diameter of the connecting tube may be from 5 mm to 6 mm.

In some embodiments, a length of the connecting tube may be from 1000 mm to 1300 mm.

In some embodiments, the fixing member may include a first fixing member and a second fixing member. The first fixing member may be configured to fix the connecting tube on an edge of the inner surface of the mask. The second fixing member may be configured to fix the first end of the connecting tube on the inner surface of the mask and make the first end of the connecting tube facing toward a breathing part of a wearer of the mask.

In some embodiments, an opening of the connecting tube at the first end may be located at a middle position of the inner surface of the mask along an up and down direction of the mask.

In some embodiments, the first fixing member or the second fixing member may be detachably connected to the mask.

In some embodiments, the first fixing member may include a flexible member and a first adhesive member disposed on one side of the flexible member, and the first adhesive member may be configured to adhere to the edge of the inner surface of the mask.

In some embodiments, the flexible member may include sponge, and the first adhesive member may include 3M glue.

In some embodiments, the flexible member may be provided with a through hole through which the connecting tube passes.

In some embodiments, the second fixing member may include a fixing body and a connecting member disposed on the fixing body, and the connecting member is connectable to the inner surface of the mask.

In some embodiments, the fixing body may be provided with an insertion hole, and a diameter of the insertion hole may be smaller than or equal to a diameter of the connecting tube at the second end.

In some embodiments, the connecting member may include a second adhesive member or a magnetic member.

In some embodiments, the fixed body may include soft PVC or silica gel.

In some embodiments, the air pump may include an air inlet, an air outlet, and a filter component disposed between the air inlet and the air outlet. The air outlet may be connected to the second end of the connecting tube.

In some embodiments, the air pump may be provided with a groove at the air inlet, and the groove may be configured to accommodate the filter component.

In some embodiments, the air filtration efficiency of the filter component may be greater than 90%.

In some embodiments, the filter component may include at least one layer of filter sheets. Materials of the filter sheets may include melt-blown cloth or activated carbon.

In some embodiments, a length of the air pump may be from 130 mm to 150 mm, a width of the air pump may be from 80 mm to 100 mm, and a thickness of the air pump may be from 40 mm to 55 mm.

In some embodiments, an intake air volume of the air pump may be from 4 L/min to 6 L/min.

In some embodiments, the air pump may further include a switch button configured to control the air pump to start working or stop working. An outer periphery of the switch button may be provided with an LED aperture that is electrically connected to the air pump, and the LED aperture may be configured to display a working state or a battery level of the air pump.

### BRIEF DESCRIPTION OF THE DRAWINGS

The present disclosure is further described in terms of exemplary embodiments. These exemplary embodiments are described in detail with reference to the drawings. It should be noted that the drawings are not to scale. These embodiments are non-limiting exemplary embodiments, in which like reference numerals represent similar structures throughout the several views of the drawings, and wherein:

FIG. 1 is a structural schematic diagram of a breathing assist device according to some embodiments of the present disclosure;

FIG. 2 is a structural schematic diagram of a second fixing member according to some embodiments of the present disclosure;

FIG. 3 is an internal structural diagram of an air pump according to some embodiments of the present disclosure; and

FIG. 4 is an external structural diagram of an air pump according to some embodiments of the present disclosure;

wherein 10 refers to a breathing assist device, 11 refers to an air pump, 12 refers to a connecting tube, 13 refers to a first fixing member, 14 refers to a second fixing member, 111 refers to an air inlet, 112 refers to an air outlet, 113 refers to a filter component, 114 refers to a groove, 115 refers to a switch button, 116 refers to a battery component, 117 refers to a coil component, 118 refers to a magnetic component, 119 refers to a gas bowl, 120 refers to a gas chamber, 121 refers to a rotating member, 122 refers to a rotating shaft, 123 refers to a heat dissipation component, 124 refers to a grating panel, 125 refers to a hook, 126 refers to a housing, 127 refers to a cover, 141 refers to a fixing body, 142 refers to a connecting member, and 143 refers to an insertion hole.

### DETAILED DESCRIPTION

In order to explain the technical solutions of the embodiments of the present disclosure more clearly, the following will briefly introduce the drawings used in the description of the embodiments. Obviously, the drawings in the following description are only some examples or embodiments of the present disclosure. For those skilled in the art, without creative work, the present disclosure may be applied to other similar scenarios according to these drawings. Unless obviously obtained from the context or the context illustrates otherwise, the same numeral in the drawings refers to the same structure or operation.

It should be understood that “system,” “device,” “unit,” and/or “module” used herein is a method for distinguishing different modules, elements, components, parts, or assemblies of different levels. However, if other words may achieve the same purpose, the words may be replaced by other expressions.

The terminology used herein is to describe particular example embodiments only and is not intended to be limiting. As used herein, the singular forms “a,” “an,” and “the” may be intended to include the plural forms as well, unless the context clearly indicates otherwise. It will be further understood that the terms “comprises,” “comprising,” “includes,” and/or “including” when used in this specification, specify the presence of stated features, integers, steps, operations, elements, and/or components, but do not preclude the presence or addition of one or more other features, integers, steps, operations, elements, components, and/or groups thereof.

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown but is to be accorded the widest scope consistent with the claims.

In daily life, air-filtering masks are widely-used respiratory protection products. People only need to wear the air-filtering masks directly on the mouth and nose to filter the air entering the mouth and nose to block harmful gases, odor, droplets, viruses, and other substances. But for some users, in particular, the elderly or people with cardiopulmonary dysfunction, wearing masks may reduce the amount of air entering the mouth and nose, resulting in breathing difficulties. In some embodiments, to solve the problem of breathing difficulties when a user wears a mask, the mask is usually connected to an air supply device (for example, an air pump, a blower, an air compressor, etc.), and the air supply device may filter and deliver outside air into the mask. When wearing the mask with the air supply device, the user inhales more air per unit time than that when only wearing an air filter mask, thereby breathing smoother. However, because the air supply device usually has a certain volume and weight, wearing the mask (with the air supply device) may cause inconvenience for the user. In addition, the mask with the air supply device is also troublesome to disinfect or replace the mask separately. Therefore, it is necessary to provide a breathing assist device for use in a mask to solve the above-mentioned problems. The present disclosure provides a breathing assist device for use in a mask. The breathing assist device mask may be applied to any type of masks (for example, ordinary masks, face masks, medical protective helmets or suits, respirators, etc.). The breathing assist device may be detachably connected to the mask so that the mask may be disinfected or replaced separately at any time for different usage scenarios. For example, when a user wears a mask connected to the breathing assist device for a time more than a specified wearing time of the mask, the user may remove the mask from the breathing assist device for disinfection and replacement. Or, when a user wearing an ordinary mask connected to the breathing assist device needs to wear a protective suit to enter a special environment (for example, a sterile workshop or a sterile ward), the user may remove the ordinary mask and connect the breathing assist device with the protective suit. As another example, when a user has a problem of breathing difficulties only during wearing a mask, the user may immediately connect the breathing assist device to the mask he/she is wearing. In addition, the breathing assist device may include an air pump and a connecting tube. The air pump may suck and filter the outside air, and then deliver the filtered air into the mask through the connecting tube, thereby solving the problem of breathing difficulties for the user. In some embodiments, the air pump may include a small or micro air pump, so that the user carries the breathing assist device when wearing a mask without causing any inconvenience.

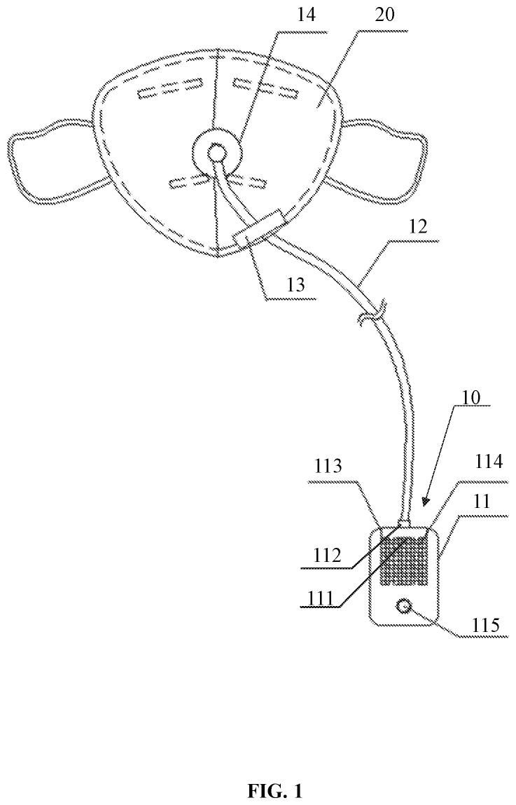

FIG. 1 is a structural schematic diagram of a breathing assist device connected to a mask according to some embodiments of the present disclosure.

In some embodiments, the breathing assist device 10 may include an air pump 11, a connecting tube 12, and a fixing member. A first end of the connecting tube 12 may be disposed on an inner surface of a mask 20 by the fixing member, and a second end of the connecting tube 12 may be connected to the air pump 11.

In some embodiments, the connecting tube 12 may be an elongated hose. In some embodiments a diameter of the connecting tube 12 may be from 5 mm to 6 mm. In some embodiments, the diameter of the connecting tube 12 may be 6 mm. In some embodiments, a length of the connecting tube 12 may be from 800 mm to 1500 mm. In some embodiments, the length of the connecting tube 12 may be from 900 mm to 1400 mm. In some embodiments, the length of the connecting tube 12 may be from 1000 mm to 1300 mm. In some embodiments, the length of the connecting tube 12 may be 1100 mm. In some embodiments, the length of the connecting tube 12 may be 1200 mm. In some embodiments, the length of the connecting tube 12 may depend on a position of the breathing assist device 10 on the user's body (for example, the chest, a shoulder, the waist, etc.). The user may shorten or bend the connecting tube 12 according to actual needs. In some embodiments, the connecting tube 12 may be made of silica gel, PVC, or a respiratory grade material (for example, food medical grade polyether-polyurethane (E-PUR)).

In some embodiments, the fixing member may include a first fixing member 13 and a second fixing member 14. The first fixing member 13 may be configured to fix the connecting tube 12 on an edge of the inner surface of the mask 20, wherein the inner surface of the mask 20 is a surface of the mask 20 facing toward the face of a wearer of the mask. The second fixing member 14 may be configured to fix the first end of the connecting tube 12 on the inner surface of the mask 20 and make the first end of the connecting tube 12 facing toward a breathing part (for example, the mouth or nose) of the wearer of the mask 20, so that the wearer may directly inhale air output from the first end of the connecting tube 12. The air output from the first end of the connecting tube 12 has been sucked by the air pump and filtered. In some embodiments, an opening of the connecting tube 12 at the first end may face toward any position on the inner surface of the mask 20. In some embodiments, the opening of the connecting tube 12 at the first end may be located at a middle position of the inner surface of the mask 20, so that the fresh air output from the opening of the connecting tube 12 at the first end may squeeze out the gas (for example, carbon dioxide) exhaled by the wear from the middle position inside the mask, and thus the wearer may better inhale the air output from the first end of the connecting tube 12, so as to better solve the problem of breathing difficulties of the wear. In some embodiments, the middle position of the inner surface of the mask 20 may be understood as a middle position along an up and down direction of the mask. In some embodiments, the middle position of the inner surface of the mask 20 may be understood as a middle position along a left and right direction. The up and down direction may be a direction parallel to a line connecting the mouth and nose of the wearer, and the left and right direction may be a direction of a line connecting the two ears.

In some embodiments, the first fixing member 13 or the second fixing member 14 may be detachably connected to the mask 20. Specifically, as shown in FIG. 1, the first fixing member 13 may include a flexible member and a first adhesive member disposed on one side of the flexible member, and the flexible member may be adhered to the edge of the inner surface of the mask 20 by the first adhesive member. When the connecting tube 12 is fixed on the edge of the inner surface of the mask 20, the connecting tube 12 may be located between the first adhesive member and the mask. In some embodiments, the connecting tube 12 may be disposed between the flexible member and the mask 20. In the process of fixing the flexible member on the edge of the inner surface of the mask 20, the flexible member may deform due to its flexibility (or elasticity) under the squeezing action between the connecting tube 12 and the flexible member, thereby forming a recess that accommodates the connecting tube 12 on the flexible member. The recess may make the connecting tube 12 not occupy extra space between the inner surface of the mask 20 and the flexible member, so that there is no gap between the flexible member and the inner surface of the mask 20 due to the connecting tube 12, which prevents unfiltered outside air from directly entering the inside of the mask through the gap between the flexible member and the inner surface of the mask 20 and from being inhaled by the wearer of the mask. The connecting tube 12 may be reliably fixed on the edge of the inner surface of the mask 20 by adhering the flexible member to the inner surface of the mask 20. In some embodiments, the flexible member may be provided with a through hole or a recessed hole. The connecting tube 12 may directly pass through the through hole or the recessed hole, so that the connecting tube 12 do not occupy extra space between the inner surface of the mask 20 and the flexible member, and thus there is no gap between the flexible member and the inner surface of the mask 20 due to the connecting tube 12, so as to prevent unfiltered outside air from directly entering the inside of the mask through the gap between the flexible member and the inner surface of the mask 20 and from being inhaled by the wearer of the mask.

In some embodiments of the present disclosure, when a user wears the mask, the flexible member may contact the user's face without causing discomfort due to the flexibility (or elasticity) of the flexible member. In some embodiments, the flexible member may include sponge, silica gel, EVA, soft PVC, or the like, or any combination thereof. In some embodiments, the first adhesive member may include a glue adhesive, a magnetic member, or a suture. The glue adhesive may include a 3M adhesive or other types of double-sided adhesives.

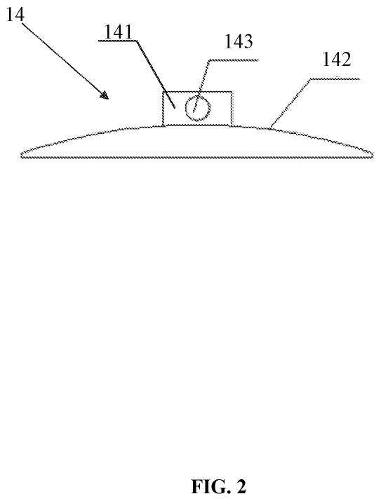

FIG. 2 is a structural schematic diagram of a second fixing member according to some embodiments of the present disclosure. As shown in FIG. 2, the second fixing member 14 may include a fixing body 141 and a connecting member 142 disposed on the fixing body 141, wherein the connecting member 142 is connectable to the inner surface of the mask 20. Specifically, the connecting member 142 may include a second adhesive member (for example, a 3M adhesive) or a magnetic member (for example, a magnetic fastener). Specifically, the second adhesive member or the magnetic member may be disposed on the connecting member 142, so that the connecting member 142 may be adhered to or adsorbed on the inner surface of the mask 20 by the second adhesive member or the magnetic member. In some embodiments, the fixing body 141 may be provided with an insertion hole 143. The insertion hole 143 may be configured to accommodate the first end of the connecting tube 12, so that the first end of the connecting tube 12 may be fixed on the inner surface of the mask 20. In some embodiments, a diameter of the insertion hole 143 on the fixing body 141 may be smaller than or equal to a diameter of the connecting tube 12, so that the first end of the connecting tube 12 may be connected to the fixing body 141 reliably after being inserted into the insertion hole 143, thereby preventing the first end of the connecting tube 12 from detaching from the insertion hole 143. In some embodiments, the diameter of the insertion hole 143 may be larger than the diameter of the connecting tube 12. In order to prevent the first end of the connecting tube 12 from detaching from the insertion hole 143, the first end of the connecting tube 12 may be fixed to the insertion hole 143 in a physical form (for example, magnetic adsorption) or a chemical form (for example, glue adhesion). In some embodiments, the fixing body 141 may be made of soft PVC, silica gel, or other flexible materials to reduce the discomfort caused when the wearer's face is in contact with the second fixing member 14. In some embodiments, the second fixing member 14 may be in a shape of a combination of a cylinder and a spherical cap as shown in FIG. 2, or in a shape of a separate cuboid, a separate cylinder, a separate spherical cap, or any irregular shape, or the like, or any combination thereof.

In some embodiments, the first fixing member 13 and the second fixing member 14 may be detachably connected to the mask 20, so that the mask 20 may be separated from or fixed to the connecting tube 12 of the breathing assist device 10 when needed, and thus the mask may be disinfected or replaced separately. In some embodiments, the breathing assist device 10 may be connected to different types of masks (for example, ordinary masks, face masks, medical protective helmets, or suits, etc.) according to the different environments where the user is located. When a user wearing an ordinary mask connected to the breathing assist device enters a special environment (for example, a sterile workshop or a sterile ward where the user needs to wear a protective suit to enter), the user may replace the ordinary mask with a protective suit and connect it with the breathing assist device 10, thereby solving the user's problem of breathing difficulties in the special environment.

In some embodiments, the connecting tube 12 may be detachably to the air pump 11, so as to facilitate the separate disinfection or replacement of the connecting tube 12. In some embodiments, the first fixing member 13 and the second fixing member 14 may have the same structure and/or material. In some embodiments, the first fixing member 13 and the second fixing member 14 may be used interchangeably. In some embodiments, one of the first fixing member 13 and the second fixing member 14 may be omitted. For example, the connecting tube 12 may be fixed on the edge of the inner surface of the mask 20 only by the first fixing member 13, while the first end of the connecting tube 12 may not be fixed. As another example, the first end of the connecting tube 12 may be fixed to the inner surface of the mask 20 by the second fixing member 14, while the connecting tube 12 may not be fixed on the edge of the inner surface of the mask 20. In some embodiments, the first fixing member 13 and the second fixing member 14 may be designed integrally with the mask 20 or the connecting tube 12. In some embodiments, the mask 20, the first fixing member 13, the second fixing member 14, and the connecting tube 12 may all be used as disposable consumables and may be replaced at any time.

See FIG. 1 again, the air pump 11 may include an air inlet 111, an air outlet 112, and a filter component 113 disposed between the air inlet 111 and the air outlet 112. The air outlet 112 may be connected to the second end of the connecting tube 12. Specifically, outside air may enter the air pump 11 through the air inlet 111 and be filtered by the filter component 113. The filtered air may exit the air outlet 112 and be delivered to the inside of the mask 20 through the connecting tube 12 for inhalation by the wearer of the mask 20.

In some embodiments, the air pump 11 may be provided with a groove 114 at the air inlet 111, and the groove 114 may be configured to accommodate the filter component 113. In some embodiments, an air filtration efficiency of the filter component 113 may be greater than 90%, where the air filtration efficiency may be understood as an ability of the filter component 113 to filter pollutants (for example, particles, viruses, or bacteria) out of the air entering the air pump. The higher the air filtration efficiency, the more pollutants may be filtered out by the filter component 113. Specifically, the filter component 113 may filter more than 90% of pollutants (for example, particles, viruses, or bacteria) out of the air entering the air pump 11. In some embodiments, the air filtration efficiency of the filter component 113 may be greater than 92%. The air filtration efficiency of the filter component 113 may be greater than 95%. The air filtration efficiency of the filter component 113 may be greater than 96%. The air filtration efficiency of the filter component 113 may be greater than 99%.

In some embodiments, the filter component 113 may include at least one layer of filter sheets. In some embodiments, materials of the filter sheets may include melt-blown cloth or activated carbon. In some embodiments, the materials of the filter sheets may also include chemical fiber non-woven filter material, glass fiber filter material, or the like. In some embodiments, the filter component 113 may include a filter sheet that meets the N95 standard (for example, the filter sheet may include melt-blown cloth used in N95 masks), whose air filtration efficiency for particles with a diameter of 0.075 μm±0.02 μm in the air is higher than 95%. In some embodiments, according to different usage scenarios, the filter component 113 may include filter sheets that meet other standards (for example, the N90 standard, the N99 standard, etc.). For example, for dust, smoke, fog, and other oily and non-oily particulate pollutants in the air, a filter sheet that meets the N90 standard may have a better air filtration efficiency. For bacteria or viruses in the air, a filter sheet that meets the N95 or N99 standard may have a better air filtration efficiency. In some embodiments, a thickness of a filter sheet may be from 1 mm to 3 mm. In some embodiments, the thickness of the filter sheet may be from 1.5 mm to 2.5 mm. In some embodiments, the filter component 113 may include 1 to 8 layers of filter sheets. In some embodiments, the filter component 113 may include 2 to 7 layers of filter sheets. In some embodiments, the filter component 113 may include 3 to 6 layers of filter sheets. In some embodiments, the filter component 113 may include 3 to 5 layers of filter sheets.

In some embodiments, the air pump 11 may include a small or micro air pump, with a relatively small volume or weight, so that the user may put the air pump 11 connected to the mask 20 into a pocket or handbag, or fix the air pump 11 on the user's portion (e.g., a shoulder, the waist) with a strap, a buckle, etc. when wearing the mask 20. In some embodiments, a length of the air pump 11 may be from 110 mm to 170 mm. In some embodiments, the length of the air pump 11 may be from 120 mm to 160 mm. In some embodiments, the length of the air pump 11 may be from 130 mm to 150 mm. In some embodiments, a width of the air pump 11 may be from 60 mm to 120 mm. In some embodiments, the width of the air pump 11 may be from 70 mm to 110 mm. In some embodiments, the width of the air pump 11 may be from 80 mm to 100 mm. In some embodiments, a thickness of the air pump 11 may be from 30 mm to 65 mm. In some embodiments, the thickness of the air pump 11 may be from 35 mm to 60 mm. In some embodiments, the thickness of the air pump 11 may be from 40 mm to 55 mm. In some embodiments, the dimension of the air pump 11 may be 130 mm×90 mm×50 mm, that is, the length, width, and thickness of the air pump 11 may be 130 mm, 90 mm, and 50 mm, respectively. In some embodiments, the dimension of the air pump 11 may be 140.7 mm×59.5 mm×49.5 mm. It should be noted that the length of the air pump 11 refers to a maximum distance between a front side surface and a rear side surface of the air pump 11 during installation, the width of the air pump 11 refers to a left side surface and a right side surface of the air pump 11 during installation, and the thickness of the air pump 11 refers to a maximum distance between a top side surface and a bottom side surface of the air pump 11 during installation.

In some embodiments, as shown in FIG. 1, the air pump 11 may further include a switch button 115 for controlling the air pump to start working or stop working. Specifically, when the user wants to inhale air by pumping air into the mask 20 through the air pump 11, he/she may press the switch button 115 to start the air pump, and press the switch button 115 again to stop the air pump 11 from delivering air to the mask 20.

In some embodiments, an outer periphery of the switch button is provided with an LED aperture (not shown in the figure) that is electrically connected to the air pump 11. The LED aperture may be configured to display a working state or a battery level of the air pump 11. In some embodiments, the LED aperture is bright, indicating that the air pump 11 is in the working state (e.g., the air pump 11 is activated). In some embodiments, the LED aperture blinks, indicating that the power of the air pump 11 is insufficient, thereby reminding the user to charge the air pump 11. In some embodiments, the LED aperture is not bright, indicating that the air pump is in a non-working state (for example, the air pump 11 stops). In some embodiments, the LED aperture may be divided into multiple segments, each of which represents a corresponding battery level of the air pump 11. For example, the LED aperture may be divided into five segments, and the battery level of the air pump 11 may be divided into five levels correspondingly. A count of bright segments of the LED aperture may correspond to a remaining battery level of the air pump 11. For example, if two segments of the LED aperture are bright, it represents remaining two levels of the battery level of the air pump 11. In some embodiments, the air pump 11 may include a display screen and a detection component. The detection component may detect the air intake volume or oxygen concentration of the air pump 11 and display it to the user in real-time through the display screen. In addition, the display screen may also display the battery level of the air pump 11. In some embodiments, the air pump 11 may further include a control button for adjusting the air intake volume of the air pump 11.

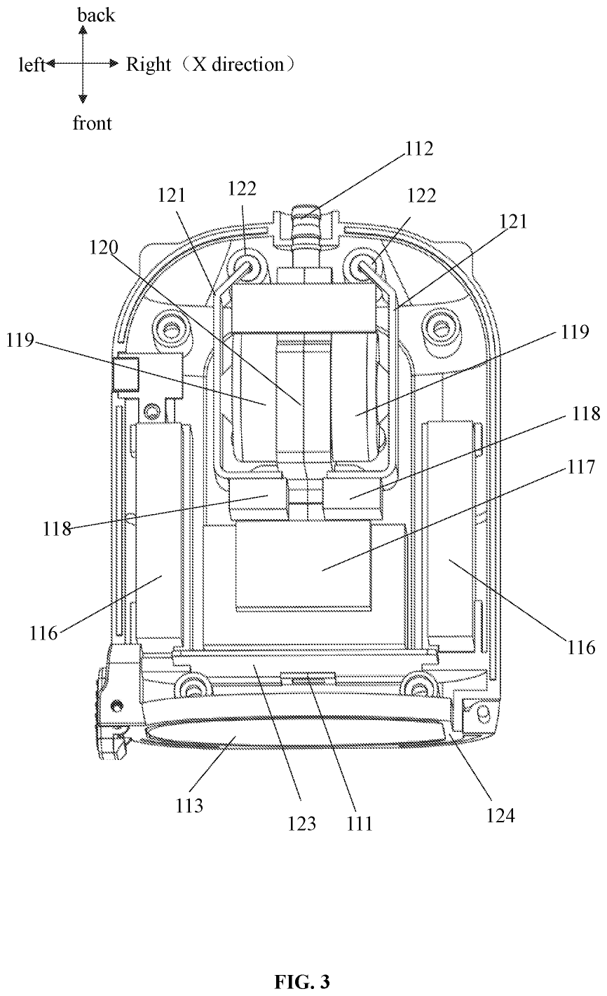

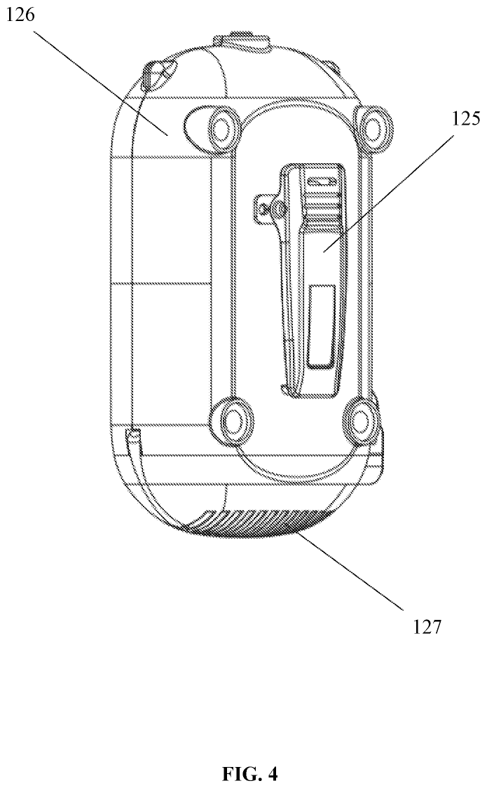

FIG. 3 is an internal structural diagram of an air pump according to some embodiments of the present disclosure. FIG. 4 is an external structural diagram of an air pump according to some embodiments of the present disclosure. The working principle of the air pump may be described in combination with FIGS. 3 and 4. It is understood that FIGS. 3 and 4 and their related descriptions are merely provided for the purpose of illustration , and not intended to limit the scope of the present disclosure.

In some embodiments, the groove 114 may be replaced by a grating panel. As shown in FIG. 3, the grating panel 124 may be disposed in front of the air inlet 111, the filter component 113 may be disposed parallel to the grating panel 124, and the air pump 11 may have a cover 127 in front of the grating panel 124 (see FIG. 4). When the cover 127 is closed, the cover 127 may directly apply pressure on the filter component 113, such that the filter component 113 can be clamped between the cover 127 and the grating panel 124. When the user opens the cover 127, the filter component 113 can be easily removed from the grating panel 124 and replaced with a new one. The outside air needs to be filtered by the filter component 113 first, then may enter the air pump 11 through the air inlet 111, and exit the air outlet 112 and be delivered to the inside of the mask 20 through the connecting tube 12. Specifically, the inside of the air pump 11 may include a battery component 116, a coil component 117, a magnetic component 118, a gas bowl 119, and a gas chamber 120. The battery component 116 may be electrically connected to the coil component 117. The gas bowl 119 and the magnetic component 118 may be jointly connected to the rotating member 121. The rotating member 121 may rotate around the rotating shaft 122. In some embodiments, when the air pump 11 is working, the battery component 116 may supply power to the coil component 117. When the coil component 117 is energized, a changing magnetic field may be generated, so that the magnetic component 118 may move to the left and right sides along the X direction shown in FIG. 3 due to the changing magnetic field, thereby driving the rotating member 121 to rotate around the rotating shaft 122. Further, the gas bowl 119 may be moved away from or close to the gas chamber 120 under the driving of the rotating member 121, wherein the gas bowl 119 and the gas chamber 120 are always adhered to increase or decrease the space between the gas bowl 119 and the gas chamber 120. When the gas bowl 119 is moved away from the gas chamber 120, the space between the gas bowl 119 and the gas chamber 120 increases, so that the air pressure therein is smaller than the pressure of outside air, and thus the outside air may enter the air pump 11 after being filtered. The air may be stored in the space between the gas bowl 119 and the gas chamber 120. When the gas bowl 119 is moved close to the gas chamber 120, the space between the gas chamber 120 and the gas bowl 119 decreases, so that the air pressure therein is bigger than the pressure of the inside of the mask 20, and thus the filtered air stored therein may exit the air outlet 112 and be delivered into the mask 20 through the connecting tube 12. In some embodiments, the battery component 116 may include a dry battery, a storage battery, a polymer battery, or the like. The magnetic component 118 may include a magnet, an energized coil, or the like.

In some embodiments, the air pump 11 may suck air from the outside mainly by the movement of the gas bowl. An air intake volume of the air pump 11 may correspond to an inner diameter of the gas bowl. The larger the inner diameter of the gas bowl, the larger the air intake volume of the air pump 11. The air intake volume of the air pump 11 refers to the volume of air sucked by the air pump 11 per unit time. In some embodiments, the air intake volume of the air pump 11 may be from 2 L/min to 8 L/min. In some embodiments, the air intake volume of the air pump 11 may be from 3 L/min to 7 L/min. In some embodiments, the air intake volume of the air pump 11 may be from 4 L/min to 6 L/min. In some embodiments, the air intake volume of the air pump 11 may be 4 L/min. In some embodiments, the air intake volume of the air pump 44 may be 5 L/min. In some embodiments, the inner diameter of the gas bowl may be from 10 mm to 40 mm. In some embodiments, the inner diameter of the gas bowl may be from 15 mm to 35 mm. In some embodiments, the inner diameter of the gas bowl may be from 20 mm to 30 mm. In some embodiments, the inner diameter of the gas bowl may be 24 mm. In some embodiments, the inner diameter of the gas bowl may be 25 mm.

As shown in FIG. 3, the air pump 11 may further include a heat dissipation component 123 (for example, a heat dissipation fin). The heat dissipation component 123 may be disposed in front of or around the coil component 116 to reduce the heat generated when the coil component 116 is energized, thereby preventing the heat of the air pump 11 from affecting user experience.

In some embodiments, the air pump 11 may also include a noise reduction component (for example, a noise-absorbing cotton or a noise-absorbing net) to reduce the sound generated by the air pump 11 during operation, respectively, thereby increasing the usage experience.

In some embodiments, as shown in FIG. 4, a hook 125 may be also disposed on the housing 126 of the air pump. The user may wear the air pump 11 on with the hook 125. For example, the air pump 11 may be hung on clothes, pants, or a pocket of the user by the hook 125, which makes it convenient for the user to carry at any time, and not easy to fall.

It should be noted that in the present disclosure, the mask 20 may include ordinary masks, glass masks, medical protective helmets or suits, respirators without breathing assist devices (or air supply devices) or respirators with breathing assist devices but having insufficient air supply function, etc. The terms “wearer,” “user,” and “wear of a mask” in the present disclosure may be used interchangeably. In addition, the mask in the embodiments of the present disclosure may be independent of the breathing assist device or part of the breathing assist device.

The possible beneficial effects of the embodiments of the present disclosure may include but are not limited to: (1) the present discloser provides a breathing assist device, which can deliver filtered air to the inside of the mask and effectively discharge exhaust gas such as carbon dioxide remaining in the mask, so as to solve the problem of breathing difficulties when a mask is worn, and is convenient for users to carry since an air pump of the breathing assist device is small; (2) the breathing assist device may be combined with a variety of respiratory protective devices (for example, masks), and the mask may be replaced at any time or disinfected separately; (3) the filter component in the air pump of the breathing assist device is easy to replace without spending much time.

It should be noted that different embodiments may produce different beneficial effects. In different embodiments, the possible beneficial effects may be one or any combination of the above, or any other beneficial effects that may be obtained.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Furthermore, the recited order of processing elements or sequences, or the use of numbers, letters, or other designations, therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software-only solution, e.g., an installation on an existing server or mobile device.

Similarly, it should be appreciated that in the foregoing description of embodiments of the present disclosure, various features are sometimes grouped together in a single embodiment, figure, or description thereof to streamline the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

In some embodiments, numbers describing the number of ingredients and attributes are used. It should be understood that in some examples, the modifier “about,” “approximately,” or “substantially” is used to modify such numbers used in the description of the embodiments. Unless otherwise stated, “about,” “approximately,” or “substantially” indicates that the number is allowed to vary by ±20%. Correspondingly, in some embodiments, the numerical parameters used in the description and claims are approximate values, and the approximate values may be changed according to the required characteristics of individual embodiments. In some embodiments, when selecting the numerical parameter, the prescribed effective digits should be considered and the general digit retention method should be adopted. Although the numerical ranges and parameters used to confirm the breadth of the ranges in some embodiments of the present disclosure are approximate values, in specific embodiments, such numerical values are set as accurate as possible within the feasible range.

For each patent, patent application, patent application publication and other materials cited in the present disclosure, such as articles, books, specifications, publications, documents, etc., the entire contents of which are hereby incorporated into the present disclosure by reference. The application history documents that are inconsistent or conflict with the content of the present disclosure are excluded, and documents that restrict the broadest scope of the claims of the present disclosure (currently or later attached to the present disclosure) are also excluded. It should be noted that if there is any inconsistency or conflict between the description, definition, and/or term usage in the attached materials of the present disclosure and the content described in the present disclosure, the description, definition and/or term usage of the present disclosure shall prevail.

At last, it should be understood that the embodiments described in the present disclosure are merely illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A breathing assist device for use in a mask, comprising an air pump, a connecting tube, and a fixing member, wherein
a first end of the connecting tube is disposed on an inner surface of the mask by the fixing member, and
a second end of the connecting tube is connected to the air pump.

2. The breathing assist device of claim 1, wherein a diameter of the connecting tube is from 5 mm to 6 mm.

3. The breathing assist device of claim 1, wherein a length of the connecting tube is from 1000 mm to 1300 mm.

4. The breathing assist device of claim 1, wherein the fixing member comprises a first fixing member and a second fixing member, the first fixing member being configured to fix the connecting tube on an edge of the inner surface of the mask, the second fixing member being configured to fix the first end of the connecting tube on the inner surface of the mask and make the first end of the connecting tube facing toward a breathing part of a wearer of the mask.

5. The breathing assist device of claim 4, wherein an opening of the connecting tube at the first end is located at a middle position of the inner surface of the mask along an up and down direction of the mask.

6. The breathing assist device of claim 4, wherein the first fixing member or the second fixing member is detachably connected to the mask.

7. The breathing assist device of claim 4, wherein the first fixing member comprises a flexible member and a first adhesive member disposed on one side of the flexible member, the first adhesive member being configured to adhere to the edge of the inner surface of the mask.

8. The breathing assist device of claim 7, wherein the flexible member comprises sponge, and the first adhesive member comprises 3M glue.

9. The breathing assist device of claim 7, wherein the flexible member is provided with a through hole through which the connecting tube passes.

10. The breathing assist device of claim 4, wherein the second fixing member comprises a fixing body and a connecting member disposed on the fixing body, the connecting member being connectable to the inner surface of the mask.

11. The breathing assist device of claim 10, wherein the fixing body is provided with an insertion hole, and a diameter of the insertion hole is smaller than or equal to a diameter of the connecting tube at the second end.

12. The breathing assist device of claim 10, wherein the connecting member comprises a second adhesive member or a magnetic member.

13. The breathing assist device of claim 11, wherein the fixing body comprises soft PVC or silica gel.

14. The breathing assist device of claim 1, wherein the air pump comprises an air inlet, an air outlet, and a filter component disposed between the air inlet and the air outlet, wherein the air outlet is connected to the second end of the connecting tube.

15. The breathing assist device of claim 14, wherein the air pump is provided with a groove at the air inlet, the groove being configured to accommodate the filter component.

16. The breathing assist device of claim 14, wherein an air filtration efficiency of the filter component is greater than 90%.

17. The breathing assist device of claim 14, wherein the filter component comprises at least one layer of filter sheets, wherein materials of the filter sheets comprise melt-blown cloth or activated carbon.

18. The breathing assist device of claim 1, wherein a length of the air pump is from 130 mm to 150 mm, a width of the air pump is from 80 mm to 100 mm, and a thickness of the air pump is from 40 mm to 55 mm.

19. The breathing assist device of claim 1, wherein an intake air volume of the air pump is from 4 L/min to 6 L/min.

20. The breathing assist device of claim 1, wherein
the air pump further comprises a switch button configured to control the air pump to start working or stop working, wherein an outer periphery of the switch button is provided with an LED aperture that is electrically connected to the air pump, the LED aperture being configured to display a working state or a battery level of the air pump.
